(19) United States
(12) Patent Application Publication
(10) Pub. No.: US 2008/0181921 A1
(43) Pub. Date: Jul. 31, 2008

(54) MULTIPLE LAYERED PIGMENTS EXHIBITING COLOR TRAVEL

(75) Inventor: Carmine DeLuca, Peekskill, NY (US)

Correspondence Address: BASF CATALYSTS LLC, 100 CAMPUS DRIVE, FLORHAM PARK, NJ, 07932, US

(73) Assignee: BASF Corporation, Florham Park, NJ (US)

(21) Appl. No.: 11/872,764

(22) Filed: Oct. 16, 2007

Related U.S. Application Data
(60) Provisional application No. 60/829,891, filed on Oct. 18, 2006.

(51) Int. Cl. C09D 11/02; C09C 1/02; C09C 1/04; C09C 1/36; A61K 8/02; C09C 1/34; C09C 1/24

(52) U.S. Cl. 424/401; 106/461; 106/425; 106/449; 106/459; 106/453; 106/450; 106/31.9; 106/428; 106/426; 106/417

(57) ABSTRACT

The present invention relates to interference pigments on the basis of multiply coated, platelet-shaped substrates which comprise at least one layer sequence comprising: (A) a metal oxide coating having a refractive index n≧2.0, (B) a colorless metal oxide or fluoride coating having a refractive index n≦1.8, (C) a nonabsorbing metal oxide coating of high refractive index, and, wherein the pigment further comprises (D) an oxide of calcium, magnesium, or zinc.

The present pigment may be used in paints, printing inks, and for producing counterfeit-protected documents of value, such as bank notes, cheques, cheque cards, credit cards, identity cards, etc.

### CROSS REFERENCE TO RELATED APPLICATIONS

This application claims the benefit pursuant to 35 U.S.C. §119(e) to U.S. provisional patent application 60/829,891, which was filed on Oct. 18, 2006 and which is incorporated herein by reference in its entirety.

### FIELD

The present application is directed to improved multiple layered pigments.

### BACKGROUND

Many pearlescent or nacreous pigments which are based on micaeous or other lamellar substrates and have been coated with a metal oxide layer exist. As a result of reflection and refraction of light, these pigments exhibit a pearl-like luster. Depending on the thickness of the metal oxide layer, they can also exhibit interference color effects. A good description of this type of pigment can be found in U.S. Pat. Nos. 3,087,828 and 3,087,829 and L. M. Greenstein, “Nacreous (Pearlescent) Pigments and Interference Pigments”, Pigment Handbook, Volume 1, Properties and Economics, 2^{nd} Edition, Edited by Peter J. Lewis (1988), John Wiley & Sons, Inc.

The pearlescent pigments most frequently encountered on a commercial basis are titanium dioxide-coated mica and iron oxide-coated mica pearlescent pigments. It is also well-known that the metal oxide layer may be over-coated. For instance, U.S. Pat. No. 3,087,828 describes depositing Fe₂O₃ onto a TiO₂ layer while U.S. Pat. No. 3,711,308 describes a pigment in which there is a mixed layer of titanium and iron oxides on the mica that is overcoated with titanium dioxide and/or zirconium dioxide.

The oxide coating is in the form of a thin film deposited on the surfaces of the mica particle. The resulting pigment has the optical properties of thin films and thus the color reflected by the pigment arises from light interference which is dependent on the thickness of the coating. Since iron oxide has an inherent red color, a mica coated with this oxide has both a reflection color and an absorption color, the former from interference, the latter from absorption of light. The reflection colors range from yellow to red and the pigments are generally referred to as “bronze”, “copper”, “russet”, etc. The pigments are used for many purposes such as incorporation in plastics and cosmetics as well as outdoor applications such as automotive paints.

Pearlescent pigments containing ferrites are also known. For example, U.S. Pat. No. 5,344,488 and DE 4120747 describe the deposition of zinc oxide onto mica platelets which had been coated with iron oxide. The U.S. patent states that in order to avoid the disadvantage of conventional zinc oxide/mica pigments, namely the tendency to agglomerate, and to obtain a pigment which had good skin compatibility, anti-bacterial action, favorable optical absorption properties and a surface color, the zinc oxide layer is applied to a previously prepared metal oxide-coated plate-like substrate. When calcined, small needle shaped crystallites are randomly distributed on the surface layer so that the zinc ferrite layer obtained is not entirely continuous. The patent states that unlike substrates covered entirely with zinc oxide in a continuous layer, the substrates covered with a layer containing crystallites show only a slight tendency to agglomeration.

Multiple layered pigments with alternating layers of high/low/high refractive indices, usually comprising metal oxides, are well known as a means of developing optically active interference pigments; i.e., interference pigments that change color at various viewing angles. Thus, e.g., a green interference pigment may move from green to blue to red relative to the viewing angle. Such pigments are described in U.S. Pat. No. 6,596,070, employing a typical layered stack comprising: (A) a coating having a refractive index n≧2.0, (B) a colorless coating having a refractive index n≦1.8, and (C) a nonabsorbing coating of high refractive index, and, if desired, (D) an external protective layer.

A particularly useful embodiment of such a multiple layered pigment is the coating of a substrate with the following layer assembly: TiO₂ or Fe₂O₃/SiO₂/TiO₂. SnO₂ can be provided on the substrate or intermediate SiO₂ layer to improve adhesion of the TiO₂ or FeO₂O₃ layer to the substrate.

As novel and unique as the above multi-layered products are, these products suffer from several disadvantages. Typically the multiple layer pigments contain large amounts of SiO₂, 40% or better based on weight of final product, which leads to agglomeration of the coated platelets, and consequently, a product with poorer color purity and overall quality. In addition, the coating stack which forms the pigment, if not formed efficiently during the metal oxide deposition will lead to poor adhesion of the juxtaposed layer, resulting in flaking off of the layer and further product degradation. Also, since the coating stack is often not mechanically or chemically stable, a final coating layer is necessary for application purposes. As a consequence, the pigment forming process becomes cumbersome, compromising efficiency and cost effectiveness since the process goes from a one to a two-step procedure.

### SUMMARY

In order to overcome the above deficiencies, a new and novel technique, with respect to interference pigments, in general, and optical variable pigments, in particular, has been developed to form highly dense, metal oxide coating stacks. By adding an alkaline earth metal to the pigment coating stack, in particular, a coating stack with alternating layers of high/low/high refractive indices, surface area values (BET) can be reduced by a factor of 2-3. This is significant because denser metal oxide coatings have increased mechanical and chemical stability. The latter adds an improved functionality, providing products that have improved stability for particular exterior applications without resorting to a final, protective coating layer. The improved mechanical stability overcomes the cracking and stripping away of the coating as it shrinks during calcining. Also, the inclusion of an alkaline earth metal allows the metal oxide coating stack to be calcined at lower temperatures, 350-850° C., to achieve the same density as found at 850-900° C. in the absence of the metals. There is a significant advantage to be able to calcine at lower temperatures without compromising the integrity or performance of the final product.

Another possible phenomenon has been noted. X-ray data shows an alteration of the mica base in the presence of magnesium after calcining at 850° C. following the post-treatment with magnesium. Thus, in addition to the advantages noted above, inclusion of at least Mg has apparently changed the nature of the substrate.

### BRIEF DESCRIPTION OF THE DRAWINGS

FIGS. 1A and 1B depict graphically X-ray diffraction patterns for three interference pigments, Inventive Examples 5 and 6 and Control 2, aligned with powder diffraction file (PDF) references for anatase (TiO₂) and hematite (Fe₂O₃). Extra peaks that occur in the samples containing magnesium are indicated by vertical dotted lines. FIG. 1A depicts the section in the 2θ range from about 30° to about 47° with three very weak extra peaks observed in samples containing magnesium. The patterns are offset vertically to improve clarity. FIG. 1B depicts the section in the 2θ range from about 13° to about 32°. The vertical dotted line indicates the cristobolite peak.

FIG. 2 depicts graphically a X-ray diffraction pattern in 2θ range from 20° to 39° for an interference pigment containing about 4% magnesium (Inventive Example 6) aligned with PDF references for crystalline silica (silicon oxide, cristobalite and zeolite) and three magnesium phases (forsterite, magnesium iron silicate and armalcolite). Extra peaks that occur in the samples containing magnesium are indicated by vertical dotted lines.

FIGS. 3A and 3B depict graphically X-ray diffraction patterns for three interference pigments, Inventive Examples 6 and 7 and Control 3. Extra peaks that occur in the samples containing magnesium are indicated by vertical dotted lines. FIG. 3A depicts the section in the 2θ range from about 30° to about 64°. The patterns are offset vertically to improve clarity. FIG. 3B depicts the section in the 2θ range from about 13° to about 32°. In both FIGS. 3A and 3B, solid vertical lines correspond to PDF references as shown in upper right corner.

FIGS. 4A and 4B depict graphically X-ray diffraction patterns for three anatase interference pigments, Inventive Examples 8 and 9 and Control 4. Vertical lines indicate peak positions for the PDF references for anatase (TiO2) and three magnesium phases (geikielite, magnesium titanium oxide and periclase). The data for Inventive Example 8, obtained at 2 second/step count rate, are scale-expanded to the intensity level of the two samples (Inventive Example 9 and Control 4) run at 10 second/step count time. FIG. 4A depicts the section in the 2θ range from about 28° to about 44°. FIG. 4B depicts the section in the 2θ range from about 44° to about 64°. Unlabeled arrows indicate peaks of interest described in the examples.

FIGS. 5A and 5B depict graphically X-ray diffraction patterns for three rutile interference pigments, Inventive Examples 10 and 11 and Control 5. Vertical lines indicate peak positions for the PDF references for anatase (TiO2), rutile (TiO₂) and three magnesium phases (geikielite, magnesium titanium oxide and periclase). The data for Inventive Example 10, obtained at 2 second/step count rate, are scale-expanded to the intensity level of the two samples (Inventive Example 11 and Control 5) run at 10 second/step count time. FIG. 5A depicts the section in the 2θ range from about 30° to about 44°. FIG. 5B depicts the section in the 2θ range from about 44° to about 64°.

FIGS. 6A, 6B and 6C depict graphically X-ray diffraction patterns for three interference pigments, Inventive Examples 7 and 11 and Control 3, as well as a mica substrate alone, Control 6. Vertical lines indicate peak positions for the PDF references for anatase (TiO₂), rutile (TiO₂), hematite (Fe₂O₃) and three magnesium phases (geikielite, magnesium titanium oxide and periclase). FIG. 6A depicts the section in the 2θ range from about 20° to about 35°. FIG. 6B depicts the section in the 2θ range from about 44° to about 64°. In FIGS. 6A and 6B, the patterns are vertically offset to improve clarity. FIG. 6C depicts the pattern for Control 6. Unlabeled arrows indicate peaks of interest.

### DETAILED DESCRIPTION

The invention provides for the use of the pigments of the invention in paints, lacquers, printing inks, plastics, ceramic materials, glasses and cosmetic formulations.

Substrates: Suitable base substrates for the multilayer pigments of the invention are firstly opaque and secondly transparent platelet-shaped substances. Preferred substrates are phyllosilicates and metal oxide-coated, platelet-shaped materials. Of particular suitability are natural and synthetic micas, talc, kaolin, platelet-shaped iron oxides, bismuth oxychloride, glass flakes, SiO₂, Al₂O₃, TiO₂, synthetic ceramic flakes, carrier-free synthetic platelets, LCPs, or other comparable materials. A preferred transparent substrate is mica.

The size of the base substrates per se is not critical and can be matched to the particular target application. In general, the platelet-shaped substrates have a thickness of between about 0.1 and about 5 μm, in particular between about 0.2 and about 4.5 μm. The extent in the two other dimensions is usually between about 1 and about 250 μm, preferably between about 2 and about 200 μm and, in particular, between about 5 and about 50 μm.

The thickness of the individual layers of high and low refractive index on the base substrate is essential for the optical properties of the pigment. As is well known in the art, the thickness of the individual layers must be precisely adjusted with respect to each other to provide interference colors.

Metal Oxide: The variation in color which results with increasing film thickness is a consequence of the intensification or attenuation of certain light wavelengths through interference. If two or more layers in a multilayer pigment possess the same optical thickness, the color of the reflected light becomes more intense as the number of layers increases. In addition to this, it is possible through an appropriate choice of layer thicknesses to achieve a particularly strong variation of the color as a function of the viewing angle. A pronounced, so-called color flop is developed. The thickness of the individual metal oxide layers, irrespective of their refractive index, depends on the field of use and is generally from about 10 to 1000 nm, preferably from about 15 to 800 nm and, in particular, about 20-600 nm.

The pigments of the invention feature a coating (A) of high refractive index in combination with a colorless coating (B) of low refractive index and located thereon a nonabsorbing coating (C) of high refractive index. The pigments can comprise two or more, identical or different combinations of layer assemblies, although preference is given to covering the substrate with only one layer assembly (A)+(B)+(C). In order to make the color flop more intense the pigment of the invention may comprise up to 4 layer assemblies, although the thickness of all of the layers on the substrate should not exceed 3 μm.

The layer (A) of high refractive index has a refractive index n≧2.0, preferably n≧2.1. Materials suitable as the layer material (A) are all materials known to the skilled worker which are of high refractive index, are filmlike and can be applied permanently to the substrate particles. Particularly suitable materials are metal oxides or metal oxide mixtures, such as TiO₂, Fe2 O₃, ZrO₂, ZnO or SnO₂, or compounds of high refractive index such as, for example, iron titanates, iron oxide hydrates, titanium suboxides, chromium oxide, bismuth vanadate, cobalt aluminate, and also mixtures or mixed phases of these compounds with one another or with other metal oxides.

The CRC Handbook of Chemistry and Physics, 63^{rd} edition reports refractive indices for the high refractive index metal oxides as follows.

| Material | Refractive Index |
|---|---|
| TiO₂ - anatase | 2.55 |
| TiO₂ - rutile | 2.90 |
| Fe₂O₃ - hematite | 3.01 |
| ZrO₂ | 2.20 |
| ZnO | 2.03 |
| ZnS | 2.38 |
| BiOCl | 2.15 |

If titanium dioxide is used, additives or other layers may be present between the substrate and titanium dioxide. Additives include rutile directors for titanium dioxide such as tin.

The thickness of the layer (A) is about 10-550 nm, preferably about 15-400 nm and, in particular, about 20-350 nm.

Colorless materials of low refractive index suitable for the coating (B) are preferably metal oxides or the corresponding oxide hydrates, such as SiO₂, Al₂ O₃, AlO(OH), B₂ O₃ or a mixture of these metal oxides. The thickness of the layer (B) is about 10-1000 nm, preferably about 20-800 nm and, in particular, about 30-600 nm.

The CRC Handbook of Chemistry and Physics, 63^{rd} edition reports refractive indices for the low refractive index metal oxides as follows.

| Material | Refractive Index |
|---|---|
| SiO₂ - amorphous | 1.46 |
| MgF₂ | 1.39 |
| Al₂O₃ | 1.76 |
| Polymers | 1.4-1.6 is typical |

Materials particularly suitable for the non-absorbing coating (C) of high refractive index are colorless metal oxides such as TiO₂, ZrO₂, SnO₂, ZnO and BiOCl, and also mixtures thereof. The thickness of the layer (C) is about 10-550 nm, preferably about 15-400 nm and, in particular, about 20-350 nm.

Coating the substrates with layers (A) and (C) of high refractive index, a layer (B) of low refractive index and, if desired, further colored or colorless coatings produces pigments whose color, gloss, opacity and angular dependence of perceived color can be varied within wide limits.

The pigments of the invention are easy to produce by virtue of the generation of two or more interference layers of high and low refractive index, precisely defined thickness and smooth surface on the finely divided, platelet-shaped substrates.

The metal oxide layers are preferably applied by wet-chemical means, it being possible to use the wet-chemical coating techniques developed for the production of pearlescent pigments.

In the case of wet coating, the substrate particles are suspended in water, and one or more hydrolysable metal salts are added at a pH which is appropriate for hydrolysis and is chosen such that the metal oxides or metal oxide hydrates are precipitated directly onto the platelets without any instances of secondary precipitation. The pH is kept constant usually by simultaneous metered addition of a base and/or acid. Subsequently, the pigments are separated off, washed and dried and, if desired, are calcined, it being possible to optimize the calcination temperature in respect of the particular coating present. In general, the calcination temperatures are between 250 and 1000° C., preferably between 350 and 900° C. If desired, following the application of individual coatings the pigments can be separated off, dried and, if desired, calcined before being resuspended for the application of further layers by precipitation.

Coating can also take place in a fluidized-bed reactor by means of gas-phase coating, in which case it is possible, for example, to make appropriate use of the techniques proposed in EP 0 045 851 and EP 0 106 235 for preparing pearl lustre pigments.

The metal oxide of high refractive index used is preferably titanium dioxide and/or iron oxide, and the metal oxide of low refractive index preferably used is silicon dioxide.

For the application of the titanium dioxide layers, preference is given to the technique described in U.S. Pat. No. 3,553,001.

An aqueous titanium salt solution is added slowly to a suspension, heated to about 50-100° C., of the material to be coated, and a substantially constant pH of about 0.5-5 is maintained by simultaneous metered addition of a base, for example aqueous ammonia solution or aqueous alkali metal hydroxide solution. As soon as the desired layer thickness of the TiO₂ precipitate has been reached, the addition of both titanium salt solution and base is terminated.

This technique, also referred to as the titration process, is notable for the fact that it avoids an excess of titanium salt. This is achieved by supplying to the hydrolysis only that quantity per unit time which is necessary for uniform coating with the hydrated TiO₂ and which can be received per unit time by the available surface area of the particles to be coated. There is therefore no production of hydrated titanium dioxide particles not precipitated on the surface to be coated.

The application of the silicon dioxide layers can be performed, for example, as follows. A potassium or sodium silicate solution is metered into a suspension, heated to about 50-100° C., of the substrate that is to be coated. The pH is held constant at about 6-9 by simultaneous addition of a dilute mineral acid, such as HCl, HNO₃ or H₂SO₄. As soon as the desired layer thickness of SiO₂ has been reached, the addition of the silicate solution is terminated. The batch is subsequently stirred for about 0.5 h.

It is now been found that pigments such as described above and, in particular, pigments formed by coating stacks comprised of alternating layers of metal oxides of high refractive index and low refractive index can be improved by the addition of alkaline earth metals or zinc. For example, calcium, magnesium or zinc can be added to the pigment after formation of the coating stack (i.e., coating layers (A), (B), and (C)). It should be noted that some alkaline earth metals, such as Be, Ba, Sr and Ra are not approved for use in cosmetics. It is part of this invention that the layers of high refractive index in the pigment do not include the same metal additive. It has been found that surface areas (BET) of the pigments as formed above can be significantly reduced and, consequently, the coating stack can be made denser providing for increased mechanical and chemical stability. Transition metals such as Fe, Cr, Mn, Co, and Cu have been previously used as additives in TiO₂ coated pigments. Such additives are not believed to have been used for densification of the final calcined layers. Thus, no substantial change in surface area values is believed to have occurred in the calcined TiO₂ layer.

Further, it has been found that the inclusion of, e.g., calcium, magnesium or zinc into the coating stack of the pigment allows the coating stack to be calcined to form the metal oxides at much lower temperatures to yield the same density as registered at higher temperatures in the absence of such added metals. The lower calcination temperatures are important in that not only is reduced energy consumed, but the integrity and performance of the pigment can be maintained. Interestingly, it has been found that alterations to the base substrate can be achieved by the post treatment addition of, e.g., Ca, Mg, or Zn in accordance with the process of the present invention. More specifically, it has been found that the presence of magnesium in the mica base has been achieved following the post treatment of the coating stack with magnesium. Thus, the process of the present invention may modify the properties of the substrate to allow tailoring of the substrate for improved properties.

In accordance with the process of this invention, improved pigments are provided by adding, e.g., calcium, magnesium, or zinc components as salts to the pigment subsequent to the formation of the coating stack of alternating high/low/high refractive index layers. Typically, the metals are applied by wet-chemical means in a slurry at room temperature and at a pH of at least 9, preferably at a pH of from about 10 to about 11. To ensure the interference pigments are optically variable, after the coating stack of alternating layers is prepared, the slurry is filtered, the resulting presscake is washed and re-slurried, for instance in fresh de-ionized water, adjusted to the appropriate pH, prior to the addition of the metal salt. Slurry temperatures up to about 80° C. are also exemplified. The specific form of, e.g., the Ca, Mg, or Zn metal salt is not believed to be critical to the invention and accordingly, water-soluble salts such as chlorides, nitrates, etc. can be utilized. In general, the amount of salt that is added is sufficient to provide a loading as metal of from up to about 10 wt. % of the pigment. Also exemplified are weights as metal up to 5% and typically from 0.4-2.5 wt. % of the pigment. Subsequent to the addition of calcium, magnesium, or zinc salt, the coating stack can then be calcined to form the metal oxides of all the metal salts. Calcination temperatures of from about 350-850° C. are useful.

Utility: The pigments of the invention are compatible with a large number of color systems, preferably from the sector of lacquers, paints and printing inks, especially security printing inks. Owing to the uncopyable optical effects, the pigments of the invention can be used in particular for producing counterfeit-protected documents of value, such as bank notes, cheques, cheque cards, credit cards, identity cards, etc. In addition, the pigments are also suitable for the laser marking of paper and plastics and for applications in the agricultural sector, such as for glasshouse films, for example.

The invention therefore also provides for use of the pigments in formulations such as paints, printing inks, lacquers, plastics, ceramic materials and glasses and for cosmetics preparations.

It is of course the case that for the various target applications the multilayer pigments can also be employed advantageously in blends with other pigments, examples being transparent and hiding white, colored and black pigments, and with platelet-shaped iron oxides, organic pigments, holographic pigments, LCPs (liquid crystal polymers) and conventional transparent, colored and black lustre pigments based on metal oxide-coated mica and SiO₂ platelets, etc. The multilayer pigments can be mixed in any proportion with customary commercial pigments and extenders.

### EXAMPLES

### Inventive Example 1 and Control 1

200 g of natural mica (average particle size 45-50 microns) was slurried with 1.0 liter de-ionized water and stirred at 250-300 rpm. At room temperature, 2.0-4.0 g of 39% FeCl₃ was added to pH 3.2. The slurry was heated to 75° C. (nucleation step). At temperature, 200.0 g of 39% FeCl₃ (38.0 g Fe₂O₃) was added at pH 3.2 at 1.0 ml/min. The pH was adjusted to 8.25 with 35% NaOH. 3000.0 g of sodium metasilicate×9H₂O (176.0 g SiO₂) was added at 5.0 ml/min, at pH 8.25. The pH was maintained with 17% HCl. 180.0 g of TiCl₄ (30.0 g TiO₂) was added at 1.5 ml/min at pH 1.9 constant (maintained with 35% NaOH). The slurry had optical variable properties (OVP), shifting color from red to gold to green in the reaction flask. The slurry was divided into two equal portions; a control, Control 1, with samples calcined at 500, 750 and 850° C. and a second portion, Inventive Example 1, post-treated with Mg as follows:

The slurry, at room temperature was adjusted to pH 11.0. 20.0 g of MgCl₂×6H₂O/100 ml de-ionized water was added at 2.0 ml/min at pH 11.0 constant (maintained with 10% NaOH). The slurry was processed and three samples were calcined at 350, 650 and 850° C., respectively. Based on recovered, calcined yield, approximately 1.0-1.5% Mg was added. The final product comprised natural mica/Fe₂O₃/SiO₂/TiO₂ and Mg. BET values at 850° C. indicated the Mg treated sample returned a coated surface approximately 3× as dense as the control at a similar temperature, with no cracking or stripping. The control exhibited both imperfections. OVP character was maintained in presence of Mg and, to some extent, color purity improved after Mg addition plus calcining.

### Inventive Example 2 and Control 1a

Inventive Example 1 was repeated but with the addition of 1.0-1.5% Ca. Similar results were noted. Control 1a was prepared by the method described for Control 1. Samples were calcined at a variety of temperatures (see Table 1).

### Inventive Example 3 and Control 1b

Inventive Example 1 was repeated but with the addition of 1.0-2.0% Zn. Similar results were noted. Control 1b was prepared by the method described for Control 1. Samples were calcined at a variety of temperatures (see Table 1).

### Inventive Example 4

As a means of further comparison, Table 1 is presented showing the effect of Ca, Mg, Zn on the optical stack with respect to surface area densification (BET) as a function of Ca/Mg/Zn content and calcining temperature vs control sample. In Table 1, the unit for BET is m²/g.

TABLE 1

| | | BET value (m²/g) | | | | | |
|---|---|---|---|---|---|---|---|
| | Calcination | | | 500° | 650° | 750° | 850° |
| | temperature | 350° C. | 450° C. | C. | C. | C. | C. |
| Control 1a | control | 10.10 | — | — | 7.49 | — | 2.10 |
| Inv. Ex. 2 | 1.3% Ca | 4.67 | — | — | 3.20 | — | 0.64 |
| Control 1 | control | — | — | 10.70 | — | 4.90 | 2.97 |
| Inv. Ex. 1 | 1.5% Mg | 4.20 | 5.90 | — | 3.27 | 2.50 | 0.70 |
| Control 1b | control | 7.55 | — | — | 5.22 | — | 2.45 |
| Inv. Ex. 3 | 2.0% Zn | 3.45 | — | — | 3.28 | — | 0.60 |

Although adding Ca, Mg or Zn results in extreme densification at 850° C., these additives advantageously give the ability to densify metal oxide surfaces at much lower than normal calcining temperatures. For example, the Ca control sample returned a BET of 7.5 m²/g at 650° C., while its Ca treated partner registered a value of 3.2 m²/g at the same temperature. Even at 350° C., the Ca coated product is much denser than its control partner. As noted, Mg and Zn treated samples behave in a similar fashion. Thus, this technique is both unique and cost-effective without compromising the OVP characteristics of the product.

Table 2 defines the OVP color shift of the above samples at 350° C. and 850° C. respectively.

TABLE 2

| | 350° C. | 850° C. |
|---|---|---|
| control | violet to orange to gold | orange to yel to y-green |
| 1.35% Ca | bl/green to red to gold | orange to gold to y-green |
| control | gold to green to bl-green | green to blue to red |
| 1.5% Mg | yel to blue to green | green to purple to red |
| control | violet to red to gold | red to gold to y-green |
| 2.0% Zn | violet to orange to gold | orange to gold to y-green |

Inclusion of Ca, Mg or Zn does not effect the OVP character of the samples but different color shifts are noted, probably as a result of degree of surface densification. In each case, quality was acceptable.

### Inventive Examples 5 and 6 and Control 2

A crystalline material is routinely identified by comparing its X-ray diffraction pattern with those of reference materials. Thus, to further characterize the interference pigments post-treated with an alkaline earth metal, X-ray diffraction data was obtained.

Control 2 was prepared by the method described for Control 1. Inventive Example 6 was prepared using the method described for Inventive Example 1 but with the addition of 4% Mg. Preparation of Inventive Example 5 was prepared using the method described for Inventive Example 1 with the following exception. Prior to the addition of magnesium (1%), the slurry containing the coating stack of alternating layers was filtered, and the resulting presscake was washed. The washed presscake was then re-slurried in fresh de-ionized water and pH adjusted to pH 11.0. The magnesium was then added as described for Inventive Example 1. Samples were calcined at 850° C.

TABLE 3

| | | Magnesium |
|---|---|---|
| | OVP stack^{a} | post-treatment |
| Control 2 | ~9% Fe₂O₃/40% SiO₂/7% TiO₂ | 0% |
| Inventive Example 5 | ~9% Fe₂O₃/40% SiO₂/7% TiO₂ | 1% |
| Inventive Example 6 | ~9% Fe₂O₃/40% SiO₂/7% TiO₂ | 4% |

^{a}Percentages are reported based on total weight of pigment.

Specimens were prepared for X-ray diffraction analysis by front loading into a deep cavity glass specimen holder, pressed with a frosted glass slide and then surface cut with a slide edge.

X-ray diffraction data were obtained by standard techniques using K-α doublet of copper radiation (at 45 kV/39 mA) and a graphite monochromator, employing 0.50, 1° and 2° DS and an 0.15 mm RS. Data collection was over the range from 7.00 to 70.0° at a 10 second/step count time.

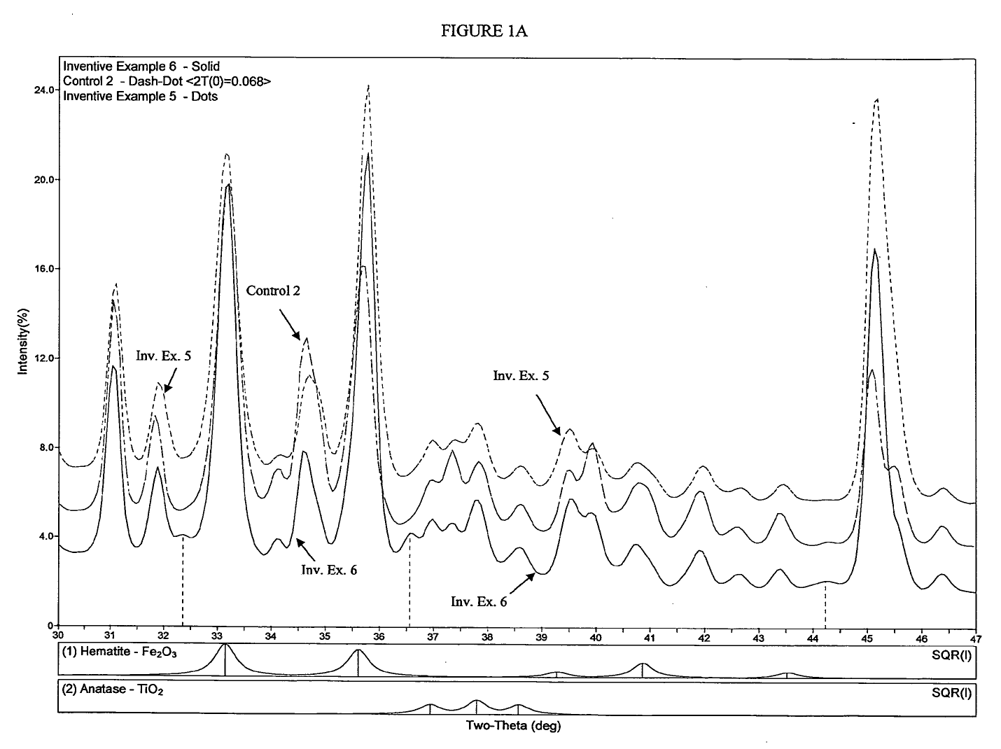

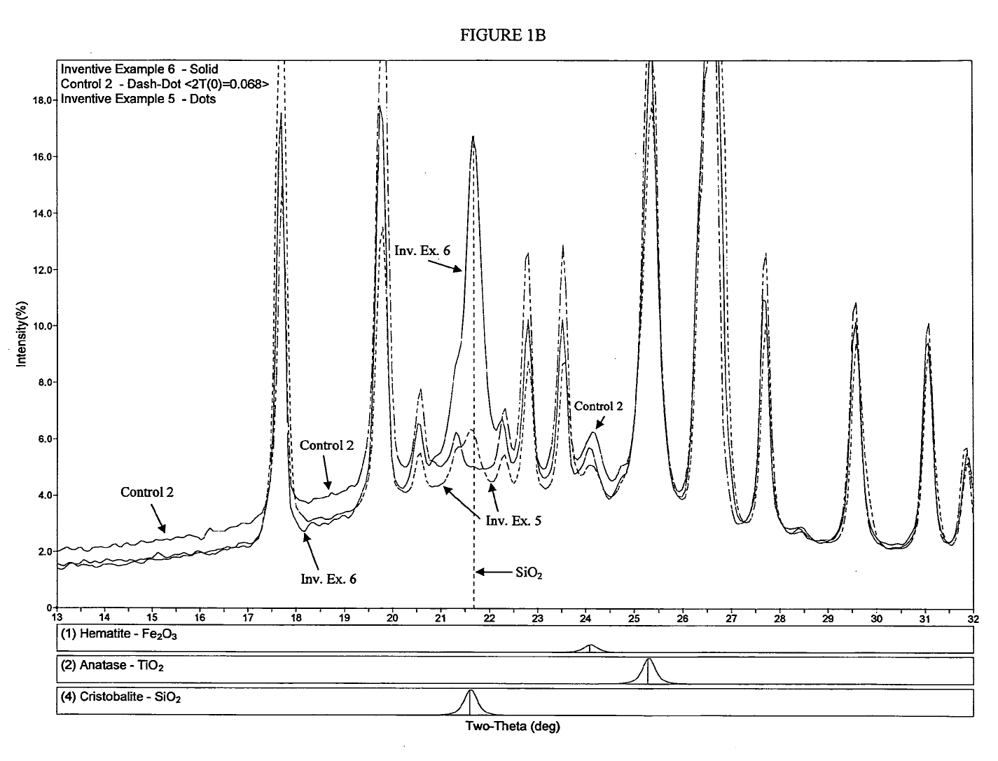

The non-mica phases present in Control 2 (pigment without magnesium) are: anatase, hematite, and likely amorphous silica. In Inventive Examples 5 and 6, the interference pigments with magnesium, these three phases also exist. However, six additional peaks were observed to be present only in the Inventive Examples, including three peaks indicated in FIG. 1A, and peaks at 21.7, 57.8, and 65.0° 2θ (not shown). The six peaks are larger in the pattern from Inventive Example 6, which had a larger amount of magnesium compared to Inventive Example 5. In addition, the amorphous band from 10°-32° centered at about 22°2θ contains less area under it in the Inventive Examples compared to Control 2 (FIG. 1B). These data suggest that crystalline silica is formed in the Inventive Examples.

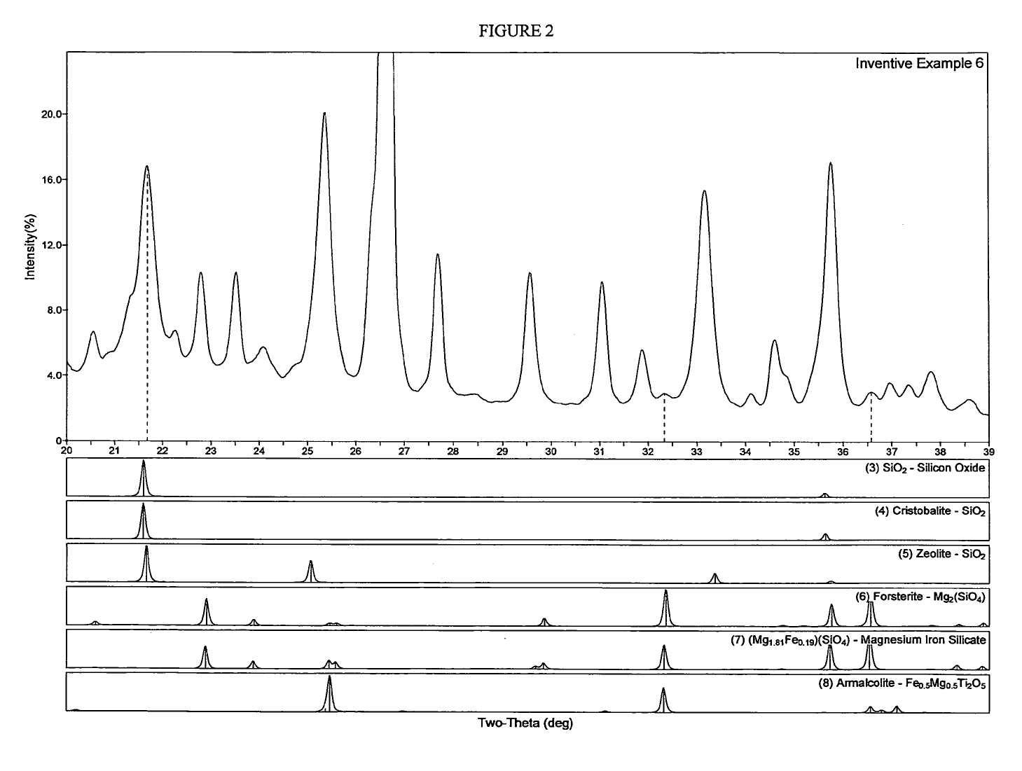

As shown in FIG. 2, the crystalline silica phase in the magnesium-containing interference pigments most resembles cristobalite, although too few peaks were observed to make a definitive determination. While three magnesium phases are possibly present, the most likely phase is magnesium silicate (forsterite), based on the matches for the three very weak peaks observed.

In conclusion, Inventive Examples 5 and 6, the pigments having magnesium post-treatment, contain two additional crystalline phases that are the same in both samples and which are not observed in Control 2. These two phases appear to be a cristobalite-type crystalline silica, SiO₂, and at least one of the following magnesium-containing phases: magnesium silicate (forsterite, Mg₂SiO₄), iron magnesium titanium oxide (amalcolite, Fe_{0.5}Mg_{0.5}Ti₂O₅), and magnesium iron silicate (olivine, Mg_{1.8}Fe_{0.2}SiO₄). The additional peaks are weaker in Inventive Example 5, which contains a smaller amount of magnesium. This result indicates that these phases are created by the magnesium. The formation of crystalline silica in the presence of magnesium, resulting in the formation of magnesium silicate, has been observed. See, for instance, Takeuchi et al., 1996, “Conversion of Silica Gel and Silica Gel Mixed With Various Metal Oxides Into Quartz,” 203: 369-374; and Zaplatyns, 1988, “The Effect of Al₂O₃, CaO, Cr₂O₃ and MgO on Devitrification of Silica,” NASA Technical Memorandum issue NASA-TM-101335, E4350, NAS1.15:101335.

Table 4 summarizes the BET surface area and color shift data for these samples.

TABLE 4

| | BET surface area (m²/g) | Color shift |
|---|---|---|
| Control 2 | 3.5648 | Red to green-gold |
| Inventive Example 5 | 1.0136 | Blue green to violet |
| Inventive Example 6 | 1.3491 | No color shift^{a} |

^{a}The addition of magnesium at pH 11, to the slurry that was adjusted from pH 1.9 to pH 11, destroyed the OVP character of the final product. Based on micro work on this sample, it appeared that some of the Ti and Si layers detached during the pH adjustment step. This observation suggests the lack of OVP character is not related to the increase in Mg. These data also support the use of the preferred two step process, wherein the slurry is filtered, washed and re-slurried prior to the addition of magnesium.

As observed for Inventive Example 1, the Mg treated products are much denser than the control sample.

### Inventive Example 7 and Control 3

To further characterize the additional crystalline phases observed in magnesium-containing interference pigments, a pigment comprising 10% magnesium was prepared and X-ray diffraction data obtained.

Control 3 was prepared using the method described for Control 1. Inventive Example 7 was also prepared using the method described for Inventive Example 1 with the following exception. Prior to the addition of magnesium (10%), the slurry containing the coating stack of alternating layers was filtered, and the resulting presscake was washed. The washed presscake was then re-slurried in fresh de-ionized water and pH adjusted to pH 11.0. The magnesium was then added as described for Inventive Example 1. Samples were calcined at 850° C.

A summary of the stack and amount of magnesium is shown in Table 5. Color shift data is shown in Table 6. The presence of OVP character in Inventive Example 7 confirms that the lack of color shift in Inventive Example 6 is not due to the amount of Mg.

TABLE 5

| | | Magnesium post- |
|---|---|---|
| | OVP stack^{a} | treatment |
| Control 3 | ~9% Fe₂O₃/40% SiO₂/7% TiO₂ | 0% |
| Inventive Example 7 | ~9% Fe₂O₃/40% SiO₂/7% TiO₂ | 10% |

^{a}Percentages are reported based on total weight of pigment.

TABLE 6

| | Color shift |
|---|---|
| Control 3 | Green to red to orange |
| Inventive Example 7 | Yellow-green to red to orange |

Specimens were prepared for X-ray diffraction analysis and X-ray diffraction data obtained as described for Inventive Examples 5 and 6 and Control 2, with the exception that data was collected over range for 2θ from 7.0° to 71.0°.

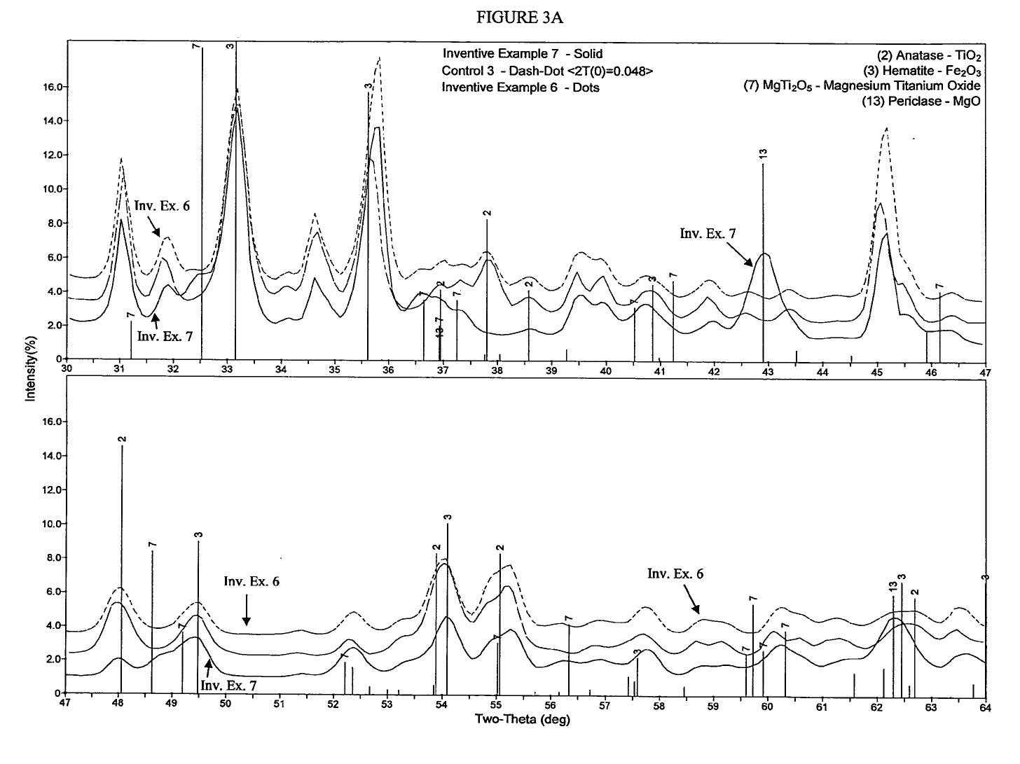

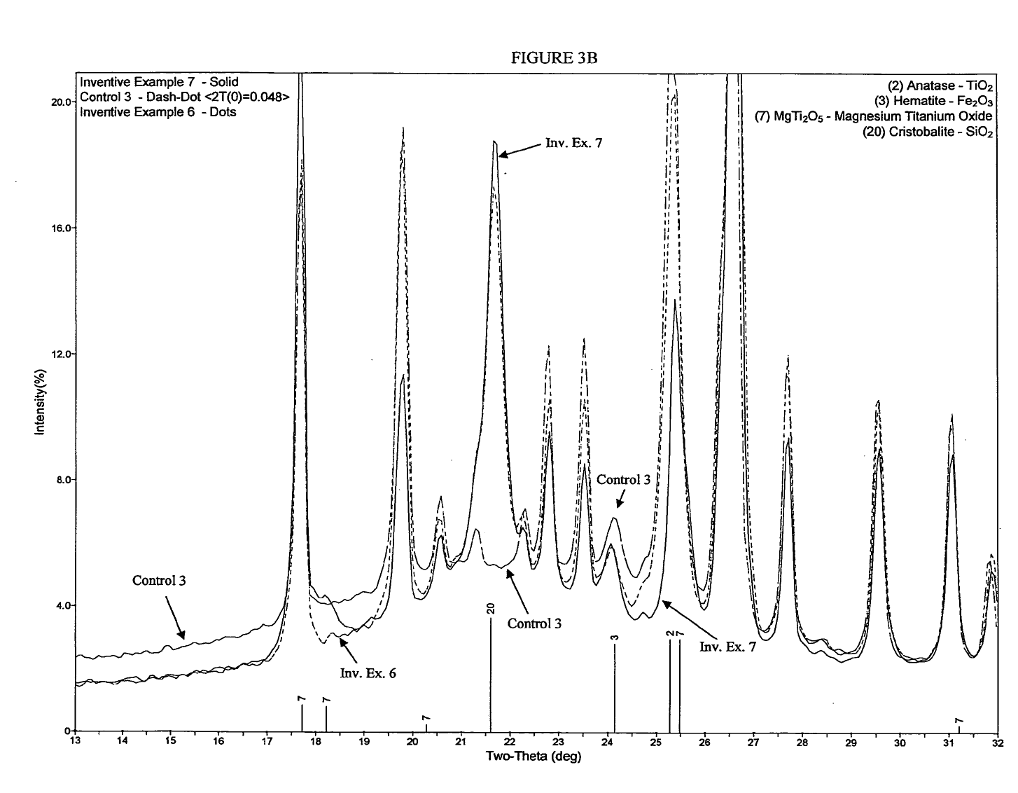

Consistent with the data for Inventive Examples 5 and 6, the additional peaks associated with the magnesium phases are larger in Inventive Example 7, the sample with more magnesium (FIG. 3A). Furthermore, size reductions in anatase peaks at 25.3, 37.8, 48.0, and 53.9° 2θ are quite evident. The amorphous band from 10-32° centered at about 22°2θ contains less area under it in the samples with magnesium than in the one without magnesium (FIG. 3B); however, the band does not change significantly with the magnesium content change. This result supports the conclusion that crystalline silica is formed in these samples and suggests that the crystallization appears to reach a limit.

Thus, the additional magnesium had a significant impact on the resulting crystalline phases present in the interference pigment. In Inventive Example 7, a very small additional amount of the amorphous silica appears to have crystallized into a cristobalite-type crystalline silica. The hematite phase appears not to have changed. Additionally, the anatase phase completely reacted with the magnesium to form magnesium titanium oxide (MgTi₂O₅); magnesium oxide (MgO) was also formed. These data also suggest that iron magnesium titanium oxide (amalcolite−Fe_{0.5}Mg_{0.5}Ti₂O₅) and magnesium iron silicate (olivine−Mg_{1.8}Fe_{0.2}SiO₄) are unlikely to be present. The magnesium silicate (forsterite−Mg₂SiO₄) phase may be present but it cannot be ascertained with certainty.

Therefore, while it is formally possible that phases change as the amount of magnesium is increased in the interference pigment, it is likely that the additional phases observed in Inventive Examples 5 and 6 (1% and 4% magnesium, respectively) are cristobalite silica and magnesium titanium oxide, MgTi₂O₅.

### Inventive Examples 8, 9, 10 and 11 and Controls 4, 5 and 6

In Inventive Examples 5-7, which are iron/silicon/titanium/mica OVP samples, it was observed that some of the amorphous silica layer crystallized into cristobalite upon addition of magnesium. To assess whether iron played a role in this crystallization, samples of Ti/Si/Ti/mica OVP were also analyzed also.

Inventive Examples 8 and 9, and Control 4 were prepared as follows. 230 g of natural mica (average particle size 45-50 microns) was slurried with 2.0 liter de-ionized water in a 5 liter Morton flask, the slurry was stirred at 300 rpm using a A410 impeller. At room temperature, the pH was reduced to 2.2 with 28% HCl. The slurry was heated to 80° C. At temperature, 200.0 g of TiCl₄ (33.3 g TiO₂) was added at pH 2.2 at 3.0 g/min. The pH was adjusted to 7.80 with 35% NaOH. 2250.0 g of sodium metasilicate×9H₂O (131.0 g SiO₂) was added at 4.0 ml/min, at pH 7.80 and 350 rpm. The pH was maintained with 28% HCl. The slurry pH was reduced to 2.2 with 28% HCl. 174.0 g of TiCl₄ (28.9 g TiO₂) was added at 3.0 g/min at pH 2.2 (maintained with 35% NaOH). A control sample was retained (Control 4).

The remaining sample was washed four times with an equal volume of DI water and the cake was reslurried in 2.0 L of DI water. The resulting filter cake was then slurried with 2 liters of DI water, the pH of the slurry was adjusted to 11.0, and the addition of the MgCl₂ solution was completed at room temperature.

Inventive Examples 10 and 11 and Control 5 were prepared as follows. 230 g of natural mica (average particle size 45-50 microns) was slurried with 2.0 liter de-ionized water in a 5 liter Morton flask, the slurry was stirred at 300 rpm using a A410 impeller. At room temperature, 42 g of 20% SnCl₄ was added at pH 1.45, at a rate of 1.0 gram/min and pH was maintained with 35% NaOH. The slurry was heated to 80° C. At temperature, 200.0 g of TiCl₄ (33.3 g TiO₂) was added at pH 1.45 at 3.0 g/min, maintained with 35% NaOH. The pH was adjusted to 7.80 with 35% NaOH. 2100.0 g of sodium metasilicate×9H₂O (123.0 g SiO₂) was added at 4.0 ml/min, at pH 7.80 and 350 rpm. The pH was maintained with 28% HCl. The slurry pH was reduced to 1.7 with 28% HCl, and 8.0 grams of 77% SnCl₄ was dumped in. The slurry was allowed to mix at 80° C. for 20 minutes without pH control. 173.0 g of TiCl₄ (28.8 g TiO₂) was added at 3.0 g/min at pH 1.45 (maintained with 35% NaOH). A control sample was retained (Control 5).

The remaining sample was washed four times with an equal volume of DI water and the cake was reslurried in 2.0 L of DI water. The slurry was mixed at room temperature and 300 rpm. To the slurry, a 1 molar solution of MgCl₂-6H₂O was added at 2.0 ml/min.

All samples were washed, filtered and calcined at 850° C. for 20 minutes

Control 6 was substrate mica material calcined at 850° C.

A summary of the stack composition, the crystalline form of titanium oxide and amount of magnesium is shown in Table 7. The weight percent for the stack is summarized in Tables 8 and 9.

TABLE 7

| | | | Magnesium |
|---|---|---|---|
| | | | post- |
| | OVP stack | | treatment |
| Control 4 | TiO₂/SiO₂/TiO₂ | Anatase | 0% |
| Inventive Example 8 | TiO₂/SiO₂/TiO₂ | Anatase | 2% |
| Inventive Example 9 | TiO₂/SiO₂/TiO₂ | Anatase | 10% |
| Control 5 | TiO₂/SiO₂/TiO₂ | Rutile | 0% |
| Inventive Example 10 | TiO₂/SiO₂/TiO₂ | Rutile | 2% |
| Inventive Example 11 | TiO₂/SiO₂/TiO₂ | Rutile | 10% |

TABLE 8

| | TiO₂ | SiO₂ | TiO₂ |
|---|---|---|---|
| Control 4 | ~8% | ~31% | ~7% |
| Inventive Example 8 | ~8% | ~30% | ~7% |
| Inventive Example 9 | ~7% | ~25% | ~6% |

TABLE 9

| | SnO₂ | TiO₂ | SiO₂ | SnO₂ | TiO₂ |
|---|---|---|---|---|---|
| Control 5 | ~0.9% | ~8% | ~30% | ~0.6% | ~7% |
| Inventive | ~0.9% | ~8% | ~28% | ~0.6% | ~7% |
| Example 10 | | | | | |
| Inventive | ~0.8% | ~7% | ~24% | ~0.5% | ~6% |
| Example 11 | | | | | |

Specimens were prepared for X-ray diffraction analysis and X-ray diffraction data obtained as described for Inventive Examples 5-7 and Controls 2 and 3 with the following exceptions. An deep cavity aluminum specimen holder was used. X-ray diffraction data was obtained at a 2 second/step count rate for samples containing 2% magnesium (Inventive Examples 8 and 10).

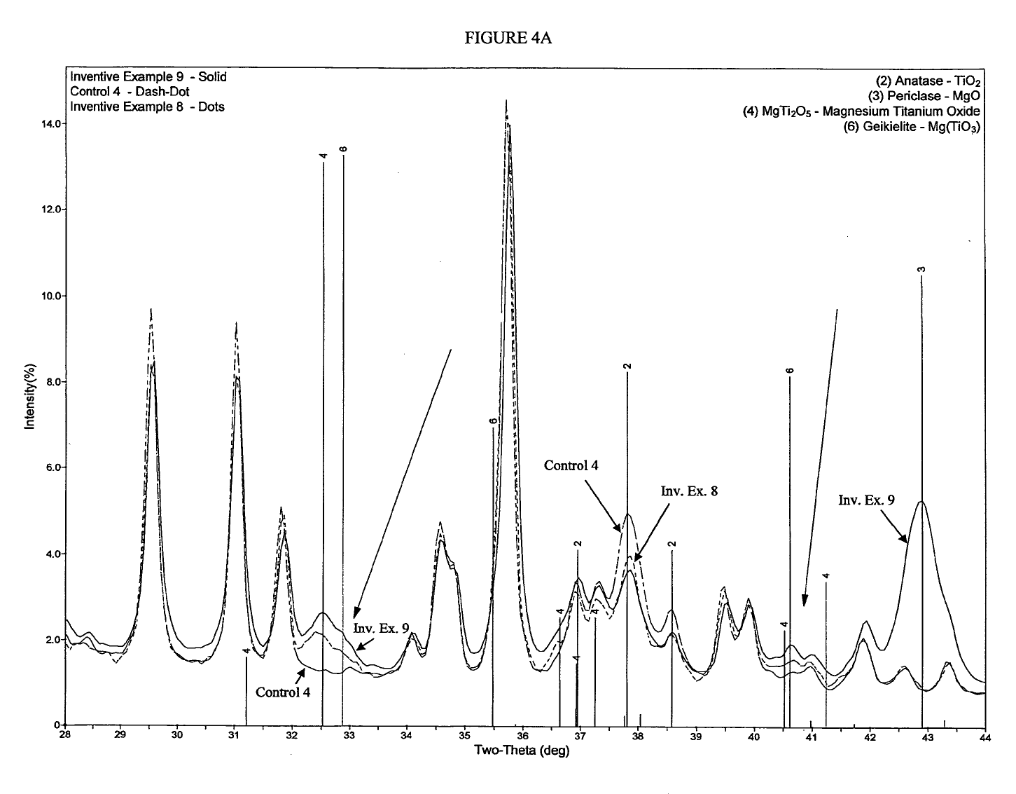

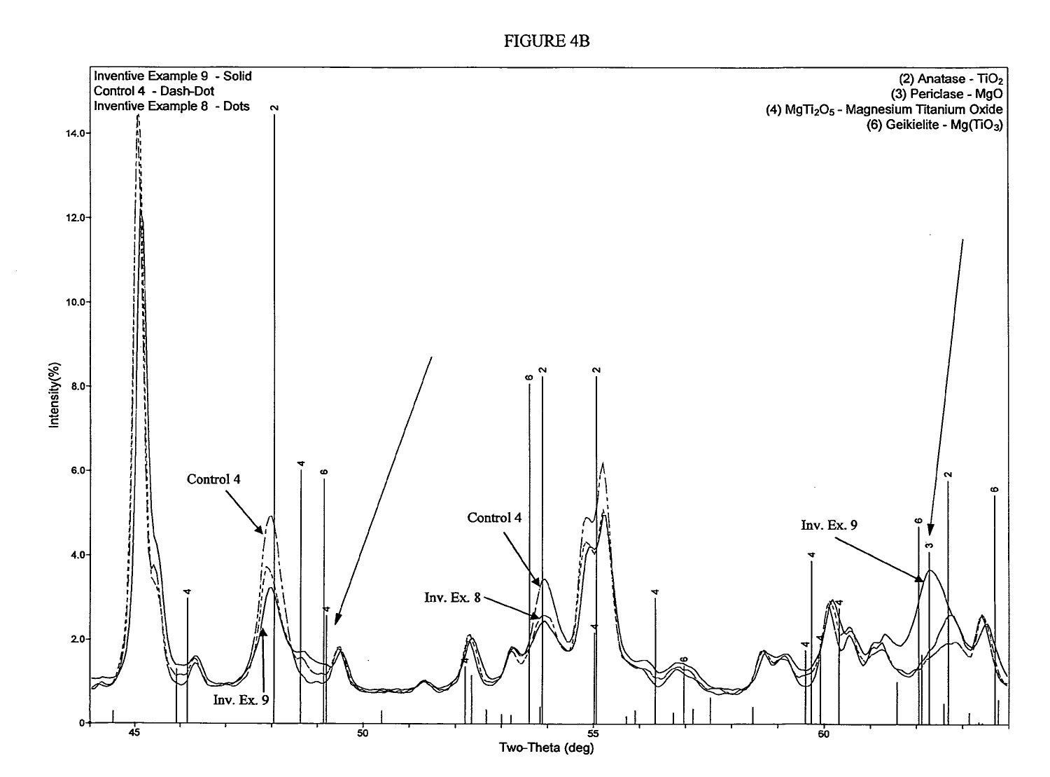

For the anatase samples, Inventive Examples 8 and 9 and Control 4, the size reductions in the anatase peaks at 25.3, 37.8, 48.0, and 53.9° 2θ are only slightly less in Inventive Example 9 (10% magnesium) compared to Inventive Example 8 (2% magnesium). See FIGS. 4A and 4B. Significant anatase was still observed (see peak at 37.8° 2θ), however, since both titania layers are anatase. No MgO was observed in Inventive Example 8 (see PDF markers labeled 3 at 42.9 and 62.4°). In addition, only a small increase in the size of the mixed oxide phase peaks (MgTiO₃, MgTi₂O₅) was observed with the increase of magnesium from 2 to 10% (compare Inventive Examples 8 and 9 peaks at 32.7 and 48.9° 2θ). This result indicates that most of the additional magnesium present in Inventive Example 9, as compared to Inventive Example 8, formed MgO.

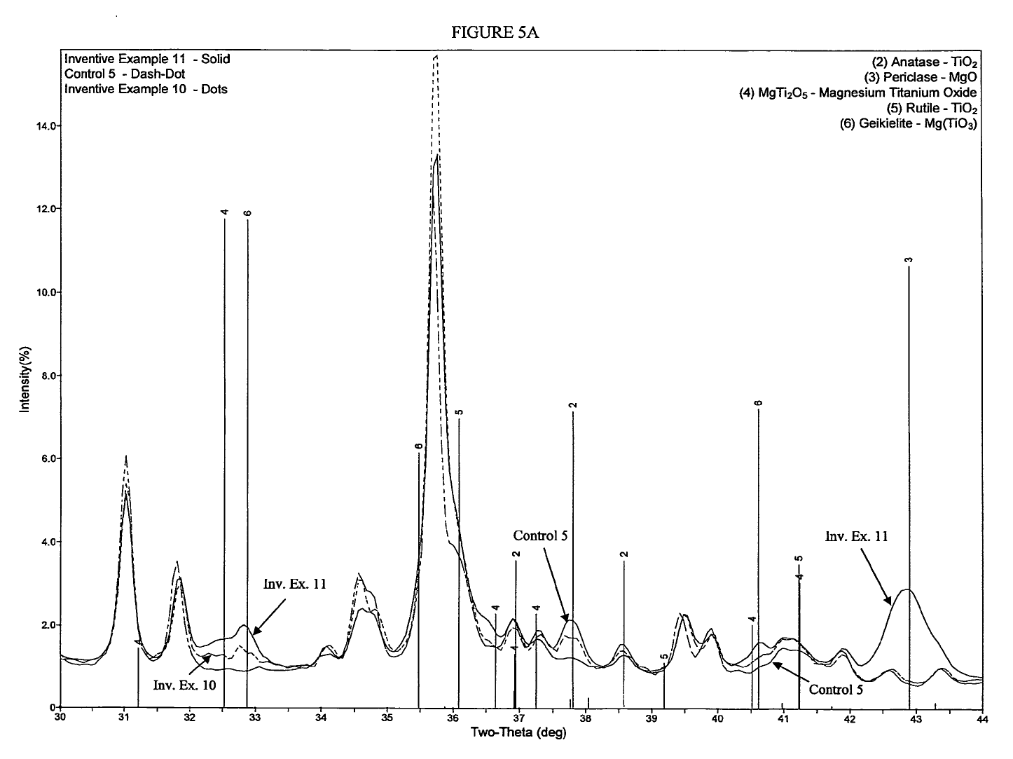

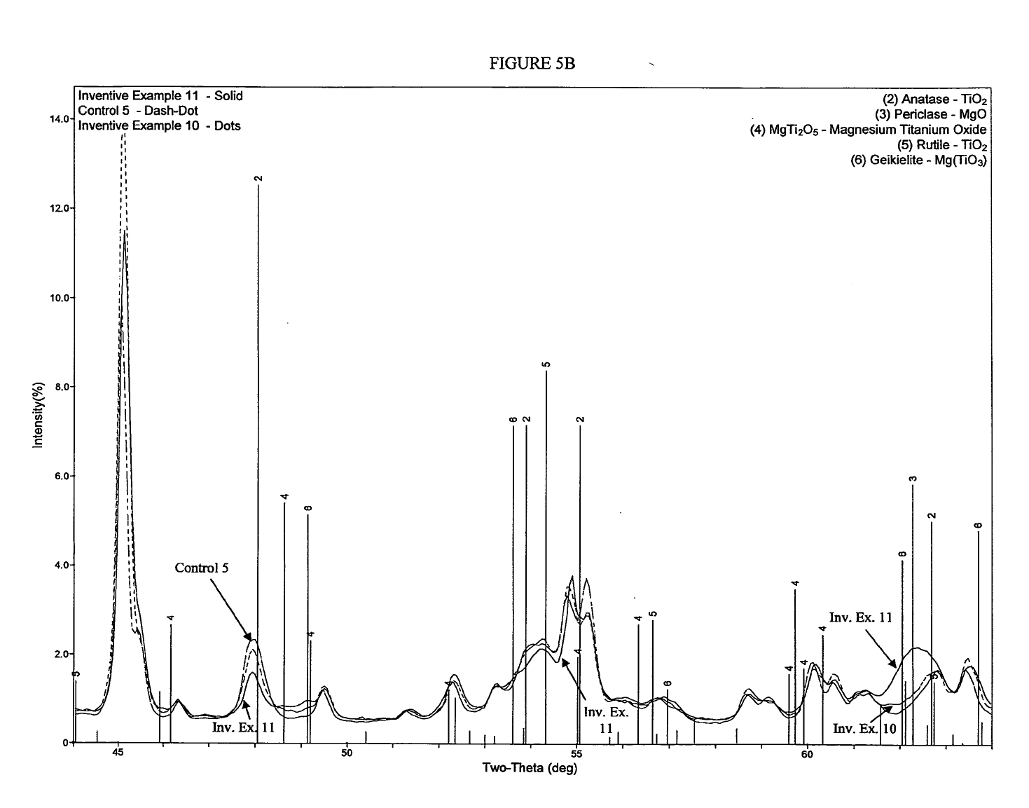

For the rutile samples, Inventive Examples 10 and 11 and Control 5, shown in FIGS. 5A and 5B, the anatase peaks were observed to almost disappear with the addition of magnesium (compare Control 5 to Inventive Example 10), however, they did not decrease markedly between the 2 and 10% magnesium (Inventive Examples 10 and 11, respectively). This result indicates the likely reason why the MgO is observed in the 10% pattern. Without wishing to be bound by theory, it is believed that the anatase in the outer TiO₂ layer reacts with the magnesium to form magnesium titanium oxide. At 10% magnesium, there is apparently insufficient anatase to react with all of the magnesium to form the mixed magnesium titanium oxide. The excess magnesium therefore forms MgO. The result is therefore believed to indicate that the inner layer of the interference pigments is most likely rutile and that most of the outer layer titania reacts with magnesium. The MgO phase was also observed to be missing from the 2% Mg sample here.

Comparison of the two sets of magnesium titanium oxide peaks at 32.7 and 48.9° 2θ in FIGS. 4A, 4B, 5A and 5B indicates that the content ratio of these two phases is different in the rutile OVP than in the anatase OVP. The significance of this difference is unknown.

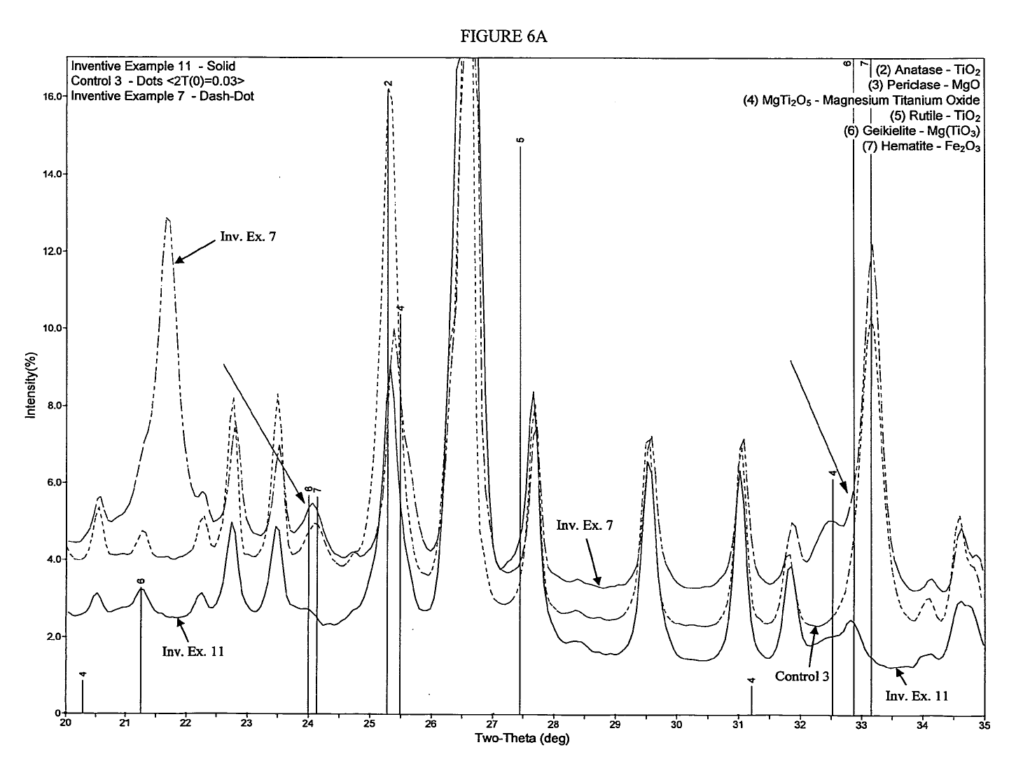

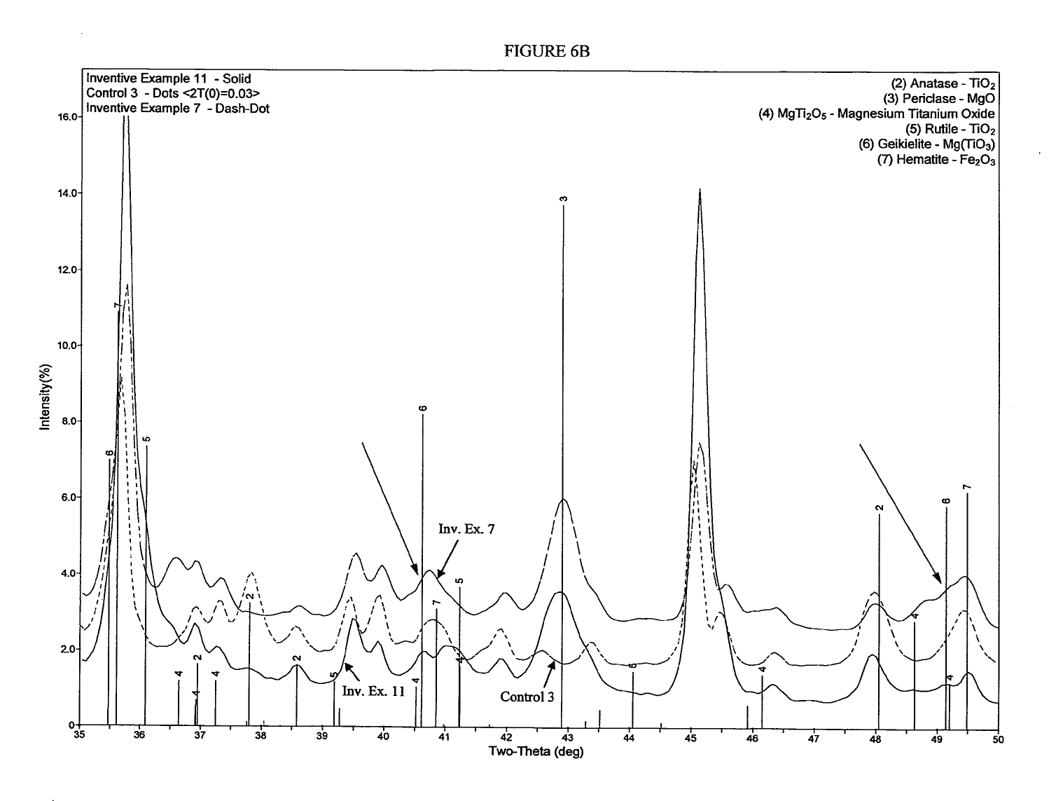

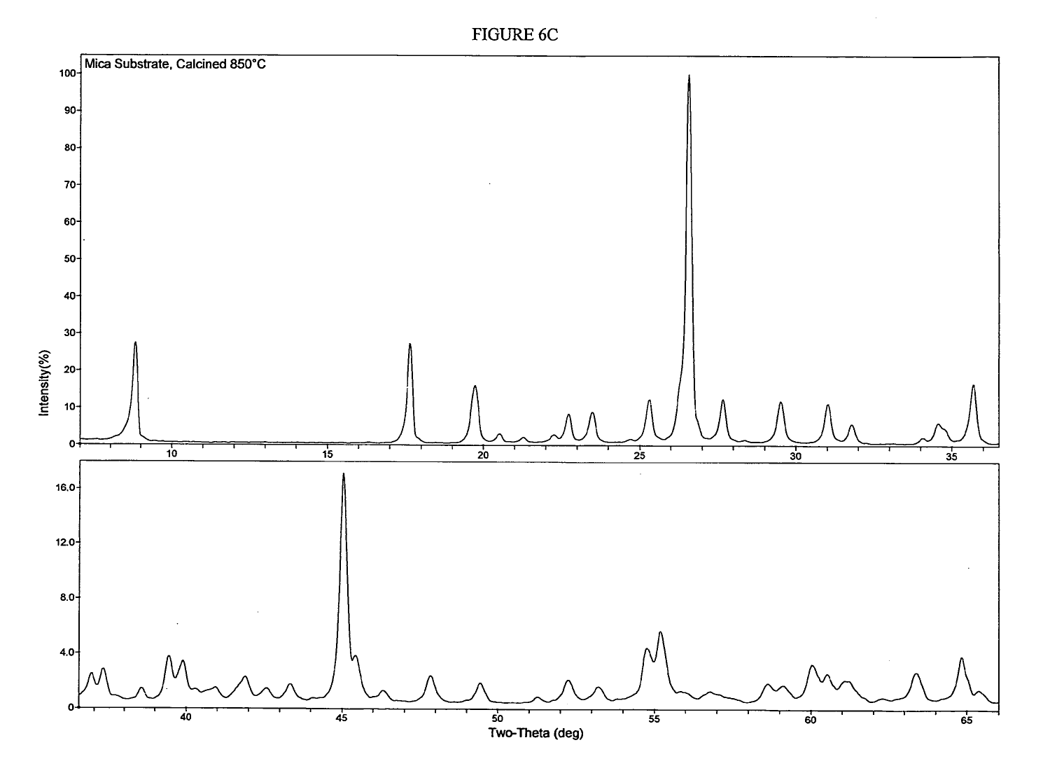

FIGS. 6A and 6B depict the X-ray diffraction patterns of Inventive Example 7 and Control 3 (Fe—Si—Ti-mica samples) and Inventive Example 11 (magnesium-containing rutile OVP sample). FIG. 6C depicts the X-ray diffraction pattern for Control 6, which is the 8500 mica reference pattern. The MgTiO₃ pattern is very similar to hematite (Fe₂O₃). A comparison of the peaks for Inventive Example 7 and those for Control 3 at 24.0, 32.8, 40.7, and 49.2° 2θ (see arrows in FIGS. 6A and 6B), however, reveals subtle shifts. These subtle peak shape differences between the with- and without-magnesium sample patterns indicate that the mixed oxide phase MgTiO₃ is likely present in the iron system sample as well.

The pattern of peaks for Inventive Example 11, the rutile OVP, illustrates the effect of the hematite interference on the MgTiO₃ peaks and the absence of the cristobalite peak at 21.6° in the rutile OVP.

In summary, anatase and rutile OVP samples were evaluated by X-ray diffraction and were observed to have slight differences in the resulting phases. To clarify the additional phases in the inventive samples at 2% magnesium samples with 10% magnesium content were prepared. The rutile OVP samples (Inventive Examples 10 and 11) contain both anatase and rutile titanium dioxide. The anatase content decreased, but the rutile content did not, probably indicating that the outer titania layer is anatase and the inner layer is rutile. The other phases formed in the magnesium-containing samples were two magnesium titanium oxide phases (MgTiO₃ and MgTi₂O₅) and in the 10% magnesium samples, magnesium oxide (MgO). That is, in the two samples to which 2% magnesium was added (Inventive Examples 8 and 10), no MgO was formed, only the two magnesium titanium oxides. The magnesium oxide phase was also not observed in the 4% magnesium sample (Inventive Example 6). Thus, greater than 4% magnesium is needed to obtain an MgO crystalline phase in the interference pigment.

These phases were also observed in the Fe/Si/Ti/Mg sample, except that the MgTiO₃ phase were not initially identified there due to interference from the hematite iron oxide pattern. Reevaluation of the Fe/Si/Ti/Mg pattern in view of the data for the Ti/Si/Ti/Mg samples suggests that the MgTiO₃ phase is most likely also present in the Fe/Si/Ti/Mg/J-mica sample.

No crystalline silica was observed in the Ti/Si/Ti/Mg samples. Thus, it appears that iron does play a role in the crystallization of the amorphous silica layer in the Fe/Si/Ti samples due to the addition of an alkaline metal.

Table 10 summarizes the crystalline phases identified in the various interference pigments.

TABLE 10

| OVP stack | Mg | Crystalline phases observed |
|---|---|---|
| Fe₂O₃/SiO₂/TiO₂ | 0% | Anatase, hematite, amorphous silica |
| | 1% | Anatase, hematite, amorphous silica, |
| | | cristobalite, MgTi₂O₅, MgTiO₃, maybe |
| | | forsterite |
| | 4% | Anatase, hematite, amorphous silica, |
| | | cristobalite, MgTi₂O₅, MgTiO₃, maybe |
| | | forsterite |
| | 10% | Hematite, amorphous silica, cristobalite, |
| | | MgTi₂O₅, MgTiO₃, MgO, maybe fosterite |
| TiO₂/SiO₂/TiO₂, | 0% | Anatase, amorphous silica |
| anatase | 2% | Anatase, MgTiO₃, MgTi₂O₅, amorphous |
| | | silica |
| | 10% | Anatase, MgO, MgTiO₃, MgTi₂O₅, |
| | | amorphous silica |
| TiO₂/SiO₂/TiO₂, rutile | 0% | Rutile, anatase, amorphous silica |
| | 2% | Rutile, anatase, MgTiO₃, MgTi₂O₅, |
| | | amorphous silica |
| | 10% | Rutile, anatase, MgO, MgTiO₃, MgTi₂O₅, |
| | | amorphous silica |

1. An interference pigment comprising a multiply coated, platelet-shaped substrate having at least one layer sequence comprising:
(A) a metal oxide coating having a refractive index n≧2.0,
(B) a colorless metal oxide or fluoride coating having a refractive index n≦1.8,
(C) a nonabsorbing metal oxide coating of high refractive index, and wherein said pigment further comprises (D) an oxide of an alkaline earth metal or zinc, or mixtures thereof wherein (D) is different from (A) and (C).

2. The interference pigment of claim 1 wherein the platelet-shaped substrate is natural or synthetic mica, glass, Al₂O₃, SiO₂ or TiO₂ flakes, or a platelet-shaped material coated with at least one metal oxide.

3. The interference pigment of claim 1 wherein the coating (A) comprises titanium dioxide, iron oxide, bismuth oxychloride, zirconium oxide, tin oxide, zinc oxide, titanium suboxides, iron titanates, iron oxide hydrates, chromium oxide, bismuth vanadate, cobalt aluminate or a mixture thereof.

4. The interference pigment of claim 1 wherein the coating (B) comprises silicon dioxide, aluminum oxide, magnesium fluoride or a mixture thereof.

5. The interference pigment of claim 1 wherein the coating (C) comprises titanium dioxide, bismuth oxychloride, zirconium oxide, tin oxide, zinc oxide or a mixture thereof.

6. The interference pigment of claim 1 containing only one layer sequence (A)-(C).

7. The interference pigment of claim 1 wherein said (D) is present in an amount of up to about 10 wt. % as metal based on the weight of the pigment.

8. The interference pigment of claim 7 wherein said (D) is present in an amount of up to about 5 wt. % as metal based on the weight of the pigment.

9. The interference pigment of claim 8 wherein said (D) is present in an amount of up to about 0.4-2.5 wt. % as metal based on the weight of the pigment.

10. The interference pigment of claim 1 wherein said coating (A) is titanium dioxide or iron oxide, (B) is silicon dioxide, and (C) is titanium dioxide.

11. The interference pigment of claim 10, wherein coating (A) is iron oxide and further wherein said pigment comprises crystalline silica.

12. The interference pigment of claim 1 wherein said (D) is calcium oxide, magnesium oxide, or zinc oxide.

13. The interference pigment of claim 12, wherein (D) further comprises magnesium titanium oxide.

14. The interference pigment of claim 1 wherein the substrate is mica.

15. A process for preparing an interference pigment of claim 1 comprising applying the metal oxides wet-chemically to the platelet-shaped substrate by hydrolytic decomposition of metal salts in an aqueous medium.

16. The process of claim 15 wherein (D) is applied subsequent to the deposition of (A), (B), and (C) and at a pH of at least 9.

17. The process of claim 16 wherein (D) is calcium oxide, magnesium oxide, or zinc oxide.

18. The process of claim 17, wherein (A) is iron oxide, and further wherein said interference pigment comprises crystalline silica.

19. The process of claim 17, wherein (D) is present in amounts of up to about 10 wt. % as metal based on the weight of the pigment.

20. A paint, lacquer, printing ink, plastic, ceramic, glass, or cosmetic formulation comprising an interference pigment of claim 1.
